(19) United States
(12) Patent Application Publication
(10) Pub. No.: US 2011/0137327 A1
(43) Pub. Date: Jun. 9, 2011

(54) SUBCUTANEOUS WAIST BAND AND METHODS RELATED THERETO

(75) Inventors: James F. Zucherman, San Francisco, CA (US); Ken Y. Hsu, San Francisco, CA (US)

(73) Assignee: SPARTEK, INC., Alameda, CA (US)

(21) Appl. No.: 12/986,904

(22) Filed: Jan. 7, 2011

Related U.S. Application Data
(63) Continuation-in-part of application No. 12/237,053, filed on Sep. 24, 2008.
(60) Provisional application No. 60/977,978, filed on Oct. 5, 2007.

(51) Int. Cl. A61B 17/12

(52) U.S. Cl. 606/151

(57) ABSTRACT

A subcutaneously placed abdominal band for waistline reduction and methods related thereto, wherein an abdominal band is subcutaneously wrapped around a person's waistline underneath the subcutaneous fat and adjacent to a facia and a muscle, the abdominal band being tightened and secured to the body by an adjustment mechanism actuatable remote from the person's body thereby tightening the person's abdominal wall and reducing the person's waistline.

### CLAIM OF PRIORITY

This application is a continuation-in-part of U.S. application Ser. No. 12/237,053, filed Sep. 24, 2008, entitled “Subcutaneous Waist Band and Methods Related Thereto” (Attorney Docket No. SPART-01033US1), which claims priority to U.S. Provisional Application No. 60/977,978, filed Oct. 5, 2007, entitled “Subcutaneous Waist Band and Methods Related Thereto” (Attorney Docket No. SPART-01033US0), all of which are incorporated herein by reference in its entirety.

### FIELD OF THE INVENTION

The present invention generally relates to a subcutaneously placed abdominal band for waistline reduction and methods related thereto.

### BACKGROUND

Numerous methods currently exist to reduce the appearance of a protruding abdomen. These methods can range from using a device to externally constrict or tighten the abdomen (such as a corset) to undergoing a major surgical procedure to alter the shape of the abdominal wall. These methods, however, have their drawbacks. For example, a person may wish to tighten his or her abdominal region in a manner which is inconspicuous, even when the person is unclothed. The use of a corset or any other exterior device to constrict the abdomen would obviously be unsuitable. The person may elect to undergo an abdominoplasty or “tummy tuck” which is a cosmetic surgery procedure used to tighten and narrow the abdominal wall. Nevertheless, a complete abdominoplasty can involve the following steps: making an incision from hip to hip just above the pubic area, making another incision to free the navel from the surrounding skin, detaching the skin from the abdominal wall from an area around the hips all the way up to the ribs (as shown in FIG. 1A), stretching the underlying muscle fascia wall and stitching it into place in its new position (as shown in FIG. 1B), tightening the remaining skin and removing the excess fat, and stitching the skin to close the wound. As is readily apparent, an abdominoplasty may be too invasive for many individuals. Accordingly, what is needed is a method to tighten the muscle and fascia of the abdominal wall that does not rely on any external devices and does not involve highly invasive surgical procedures.

### BRIEF DESCRIPTION OF THE DRAWINGS

The accompanying drawings, which are incorporated into and constitute a part of this specification, illustrate one or more embodiments and, together with the detailed description, serve to explain the principles and implementations of the invention. In the drawings:

FIGS. 1A and 1B are front views of a human torso illustrating the operative techniques used during a typical abdominoplasty in the prior art.

FIG. 2A is a rear view of a human torso illustrating an incision in the skin.

FIG. 2B is a front view of a human torso illustrating two incisions in the skin.

FIG. 2C is a side view of a human torso illustrating an incision in the skin.

FIG. 2D is a front view of a human torso illustrating a peri-umbilical incision in the skin curved cephalad to the umbilicus.

FIG. 3 is a cross-sectional view of the skin, fat, fascia and muscle along an abdominal wall.

FIG. 4 is a cross-sectional view of the skin, fat, fascia and muscle along an abdominal wall, wherein dissection of a plane of dissection adjacent to the abdominal wall has been initiated.

FIG. 5A is a rear view of a human torso illustrating the use of a curved probe to dilate the plane of dissection.

FIG. 5B is a front view of a human torso illustrating the use of a curved probe to dilate the plane of dissection.

FIG. 5C is a side view of a human torso illustrating the use of a curved probe to dilate the plane of dissection.

FIG. 6A illustrates a perspective view of a human torso wherein a plane of dissection has been created around the abdomen below the umbilicus.

FIG. 6B illustrates a perspective view of a human torso wherein a plane of dissection has been created around the abdomen above the umbilicus.

FIG. 6C illustrates a perspective view of a human torso wherein a plane of dissection has been created around the abdomen above and below the umbilicus.

FIG. 7A illustrates a rear view of a human torso wherein a band is being implanted subcutaneously for waistline reduction.

FIG. 7B illustrates a side view of a human torso wherein a band has been implanted subcutaneously for waistline reduction.

FIG. 8A illustrates an embodiment of a band to be implanted around an abdomen for waistline reduction.

FIGS. 8B-8N illustrate different embodiments of fasteners used on a band to be implanted around an abdomen for waistline reduction.

FIGS. 9A-12B illustrate alternative embodiments of adjusters used on a band to be implanted around an abdomen for waistline reduction allowing for remote adjustment of the effective band length.

FIGS. 13-14 illustrate embodiments of systems usable with adjusters and bands to be implanted around an abdomen for waistline reduction allowing for remote adjustment of the effective band length.

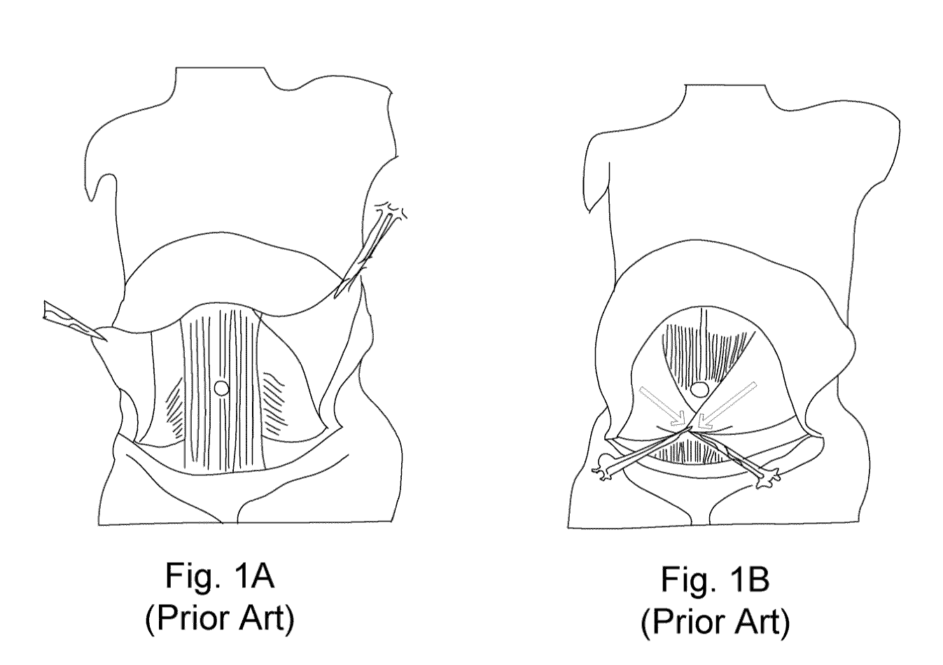

### DETAILED DESCRIPTION

Embodiments are described herein in the context of a subcutaneously implanted abdominal band for waistline reduction and methods related thereto. Those of ordinary skill in the art will realize that the following detailed description is illustrative only and is not intended to be in any way limiting. Other embodiments of the present invention will readily suggest themselves to such skilled persons having the benefit of this disclosure. Reference will now be made in detail to implementations of embodiment of the present invention as illustrated in the accompanying drawings. The same reference indicators will be used throughout the drawings and the following detailed description to refer to the same or like parts.

One of the objects of the invention is to provide a band (or “belt”) which can be surgically implanted subcutaneously to tighten the muscle and fascia of the abdominal wall. Another object of the invention is to provide a minimally invasive method of tightening the muscle and fascia of the abdominal wall using the above mentioned subcutaneous band. Other objects of the invention will become apparent from time to time throughout the specification and claims as hereinafter related.

These and other objects of the invention can be accomplished by surgically implanting a band subcutaneously around a person's waist (or “midline”) to tighten the muscle and fascia of the abdominal wall. The method of implanting the band can briefly be described as follows. First, at least one incision is made to a predetermined location around a person's waist, the incision extending through the subcutaneous fat and superficial to the fascia and muscle. Next a plane of dissection (or “plane” or “path”) is developed around the person's waist underneath the point or points of incision. The plane is developed adjacent to the fascia and muscle tissue layer wherein the fat layer is separated from the fascia and muscle tissue. Once the plane is developed, a first end of a band is inserted into the plane through an incision and wrapped around the person's waist until the first end of the band reaches the original insertion point. The first end of the band can then be withdrawn from the plane of dissection through the incision. The band can then be tightened and tied anteriorly. Finally, the band is subcutaneously secured to the body adjacent the facia and muscle whereby the abdominal wall is tightened by the band.

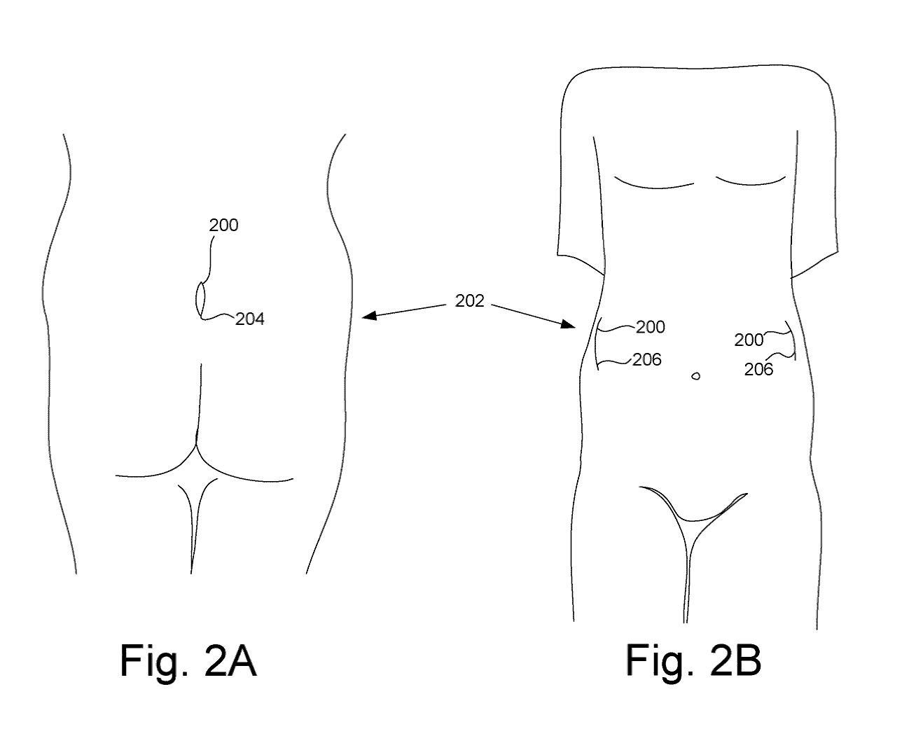

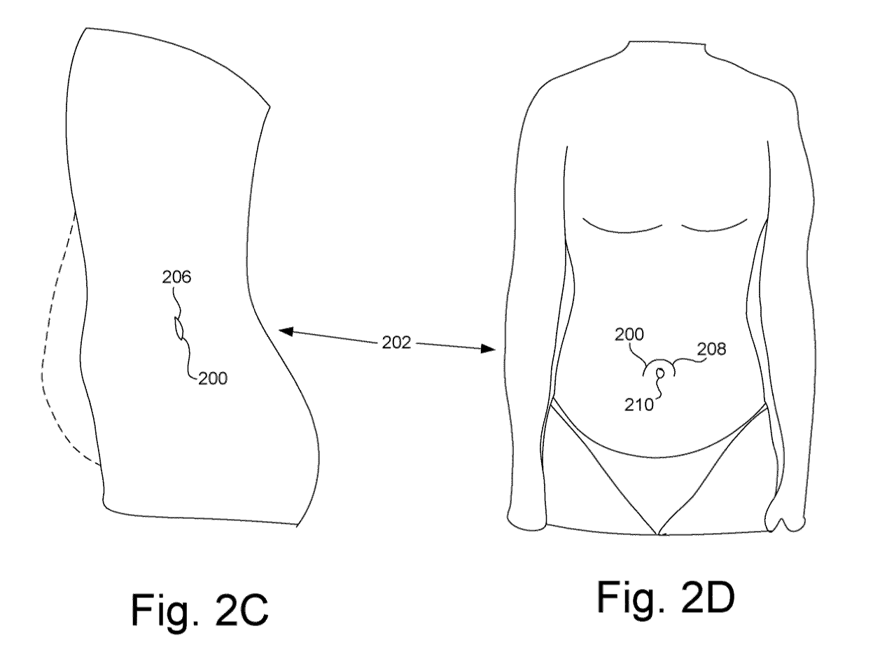

The method of implanting the band will now be described in greater detail. Referring now to FIGS. 2A, 2B, 2C and 2D, the method is commenced by making one or more incisions 200 to the person's midline 202 at predetermined locations. In an embodiment, three skin incisions 200 can be made along the person's midline 202, one incision 200 being a posterior 204 incision 200 (as shown in FIG. 2A) and the other two incisions 200 being lateral 206 incisions 200 (as shown in FIGS. 2B and 2C). In an embodiment, a peri-umbilical 208 incision 200 curved cephalad to the umbilicus 210 (as shown in FIG. 2D) may be made in addition to, or in lieu of, the posterior 204 and lateral 206 incisions 200. The length of the incisions 200 can vary depending on the width of the band being implanted in the patent. In an embodiment, the length of the incision 200 can range from one to three inches. It is noted that any number of incisions having any length may be made to the person without deviating from the scope of this invention.

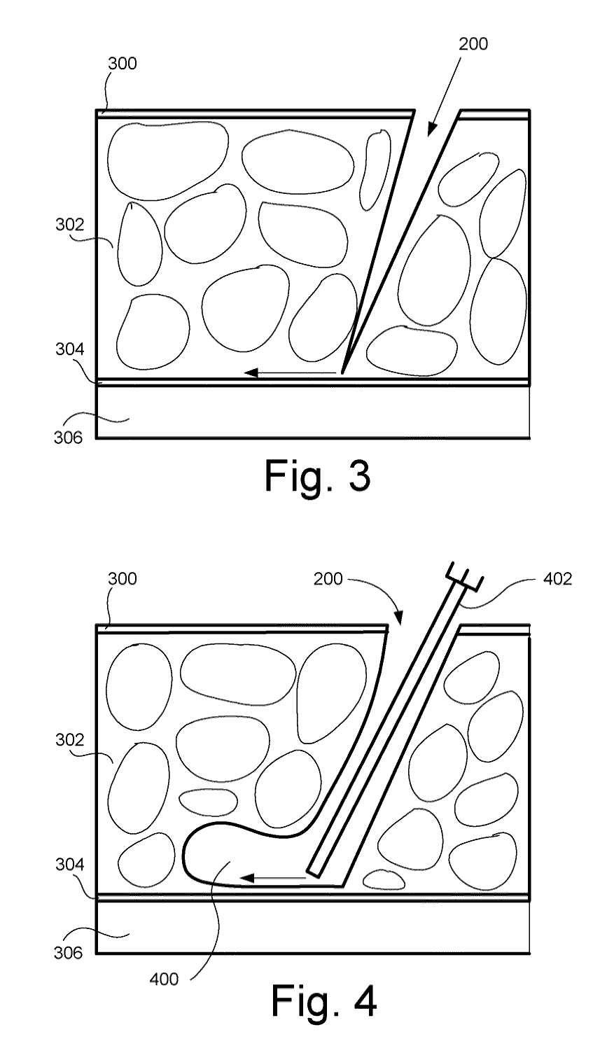

Referring to FIG. 3, the incision 200 is made through the skin 300 and underlying subcutaneous fat 302 and superficial to the fascia 304 and muscle 306. It is preferable that the incision 200 extend completely through the subcutaneous fat 302 to the fascia 304 and muscle 306. This may be accomplished by making an initial incision 200 with a scalpel and then cutting the subcutaneous fat 302 with dissecting scissors until a path to the fascia 304 and muscle 306 is created.

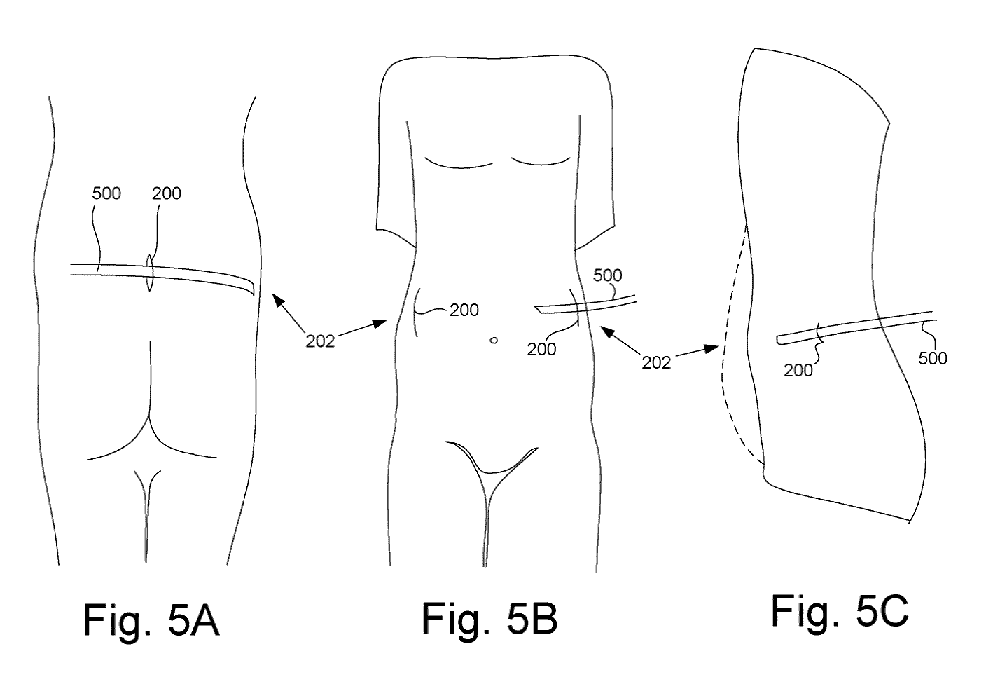

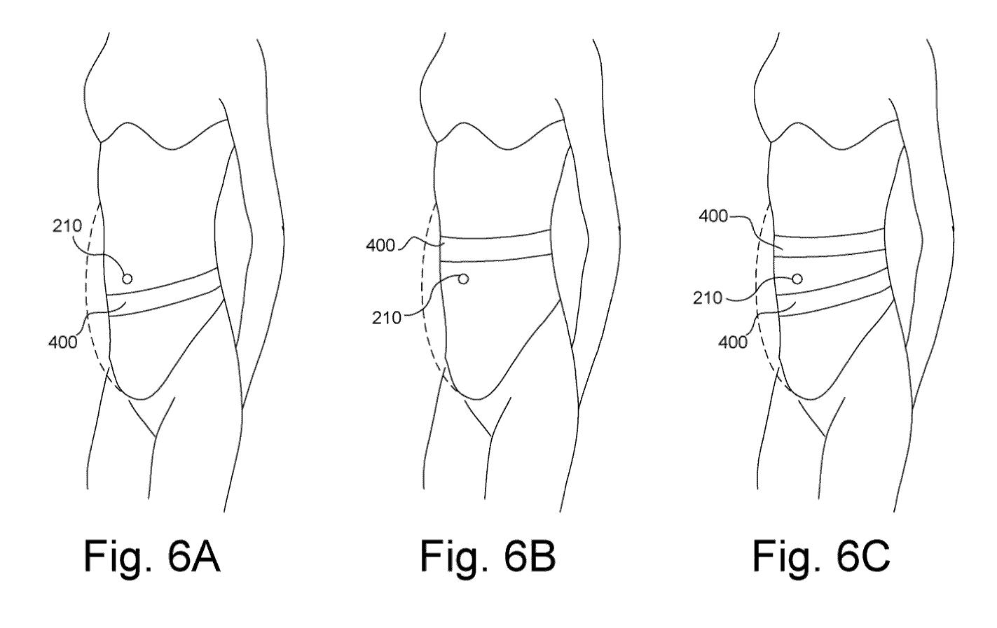

Referring to FIG. 4, after the one or more incisions 200 are made, a plane of dissection 400 can be developed deep to the subcutaneous fat 302 and superficial to the fascia 304 and muscle 306 (shown in FIG. 3) around the person's midline 202 (shown in FIG. 2A). Since fat trapped under pressure can necrose, the plane of dissection 400 is preferably located beneath all subcutaneous fat 302 adjacent to the fascia 304 and muscle 306. In an embodiment, a tool for dissection 402 is inserted into an incision 200 and advanced alongside the person's midline 202 (shown in FIG. 2A) to dissect the structure along the plane of dissection 400. In an embodiment, endoscopic dissection methods can be used. The endoscopic dissection methods can include, but are not limited to, hydro dissection, balloon dissection, ultrasonic dissection, and laser dissection may be employed to dissect the structure along the plane of dissection 400. Endoscopic dissection allows the surgeon to accurately visualize the correct plane of dissection 400 adjacent to the surface of fascia 304 and muscle 306 to ensure that it is devoid of subcutaneous fat 302. As illustrated in FIGS. 5A, 5B and 5C, a curved probe 500 may also be inserted into the one or more incisions 200 along the plane of dissection 400 (not shown) to dilate the path. A surgeon may wish to use smaller probes 500 initially, followed by larger probes 500 during this process. In an embodiment, the plane of dissection 400 can be made below the umbilicus 210 as illustrated in FIG. 6A. In another embodiment, the plane of dissection 400 can be made above the umbilicus 210 as illustrated in FIG. 6B. In yet another embodiment, the plane of dissection 400 can be made both below and above the umbilicus 210 as illustrated in FIG. 6C.

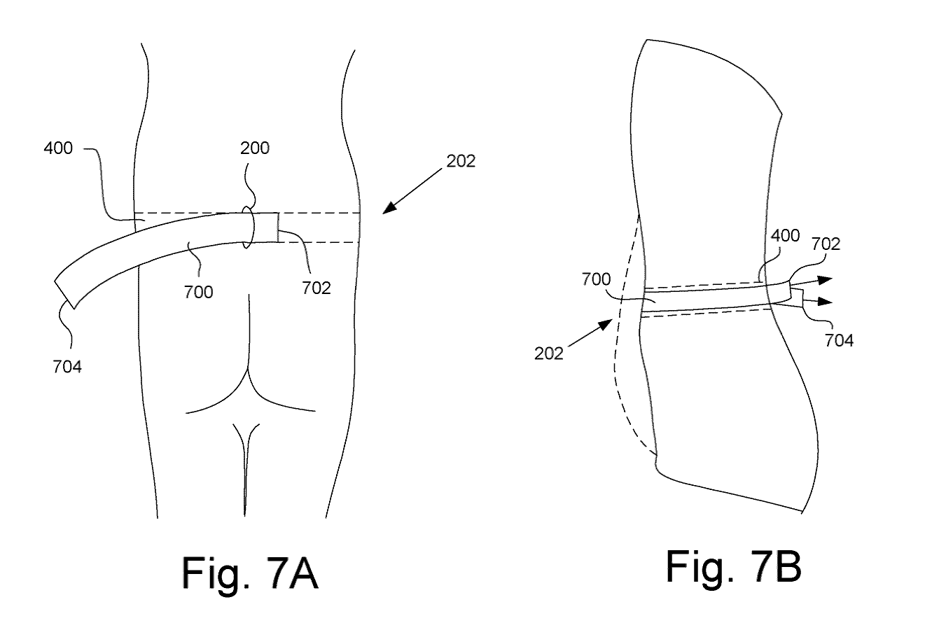

Referring to FIG. 7A, once the plane of dissection 400 has been defined, a band 700 including a first end 702 and a second end 704 may be implanted into the person. When implanting the band 700, the first end 702 of the band is passed through an incision 200 and into the plane of dissection 400. The first end 702 of the band 700 is wrapped around the person's midsection 202 within the plane of dissection 400 until the first end 702 of the band 700 reaches the original insertion point. The first end 702 of the band 700 can then removed from the plane of dissection 400 as shown in FIG. 7B. Once the first end 702 of the band 700 is removed from the plane of dissection 400, the band 700 can be anteriorily tightened and the two ends 702, 704 of the band 700 can be fastened, tied, clipped, clamped and/or otherwise connected to each other to secure the band 700 to the body. In an embodiment, the band 700 can be pulled through the plane of dissection 400 by pulling on a wire, suture or strap attached to the first end 702 of the band 700. In another embodiment, the band 700 is pushed through the plane of dissection 400. The band 700 can be pushed or pulled using a suture passer. Once the band 700 is tightened and tied, the band 700 is secured to the body and the incisions are stitched closed while the band remains wrapped around the person's abdomen.

Different embodiments for the band 700 generally described above can also be utilized. The band 700 is preferably 20 to 30 inches long (generally 20 to 40 or 50 inches long), preferably at least 1.25 inches wide (generally 1 inch to 5 inches wide or of varying width around the length as for example in the shape of a cummerbund) and preferably 1 to 3 mm thick. The band 700 is preferably constructed of a material which is biocompatible, nonresorbable and nonbiodegradable. Specific materials which may be used to construct the band 700 include, but are not limited to, dacroncrystalline polypropylene, polyethylene, polyester fiber, PLLA, PDLA, polyurethane, nylon, titanium mesh, silicon, silastic and other polymers. In an embodiment the band 700 can be elastic and/or reversible.

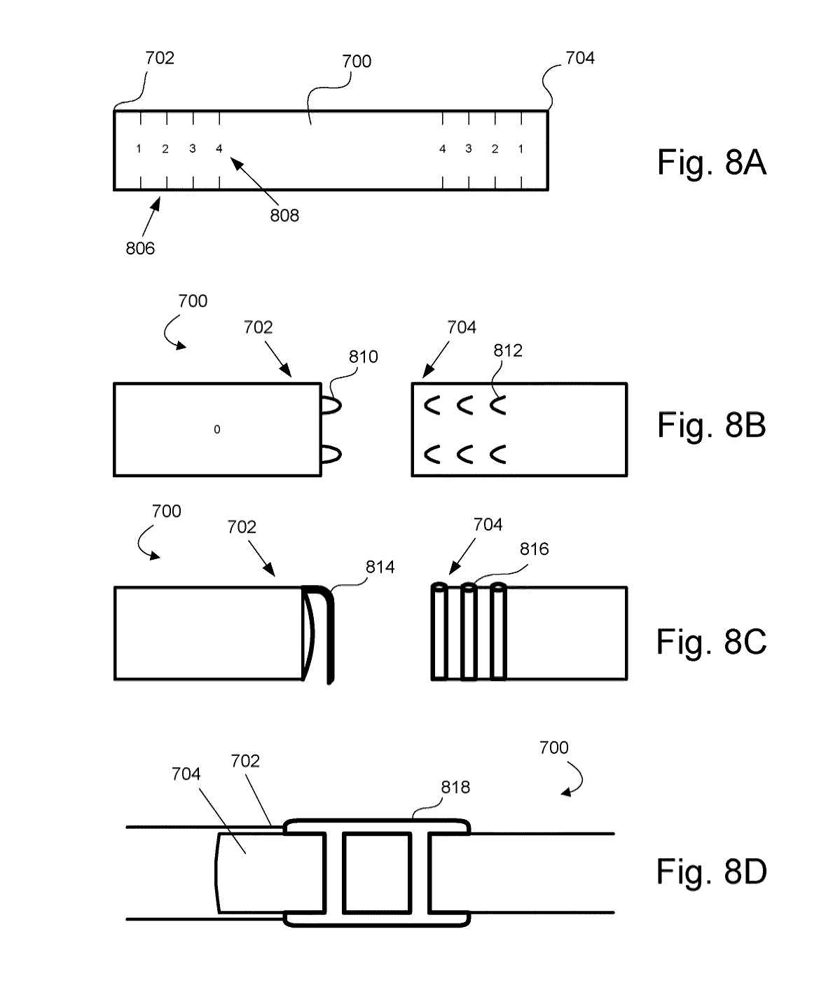

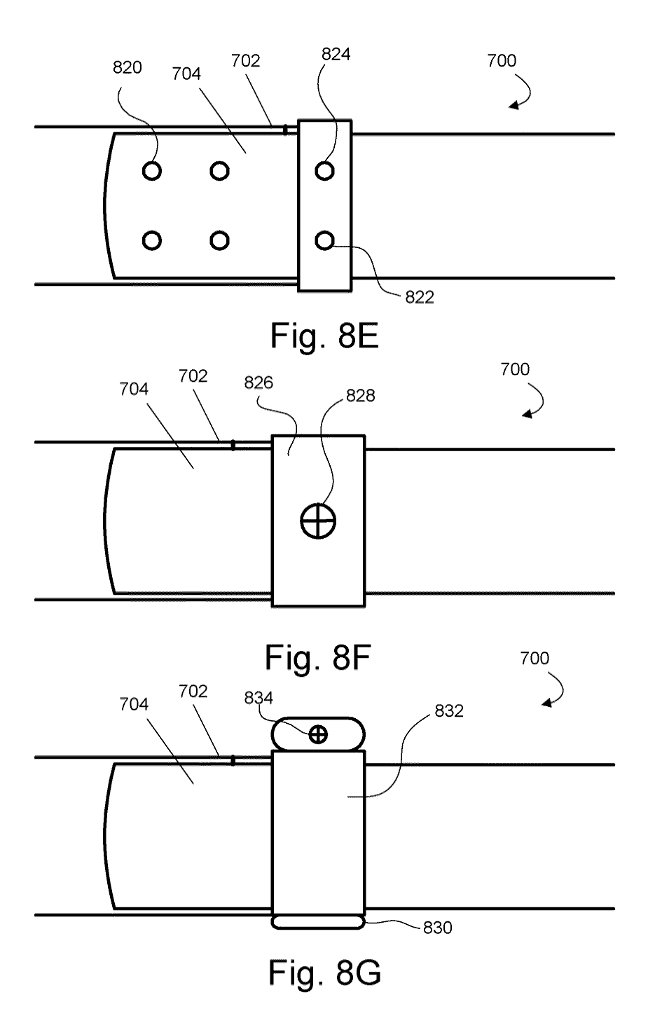

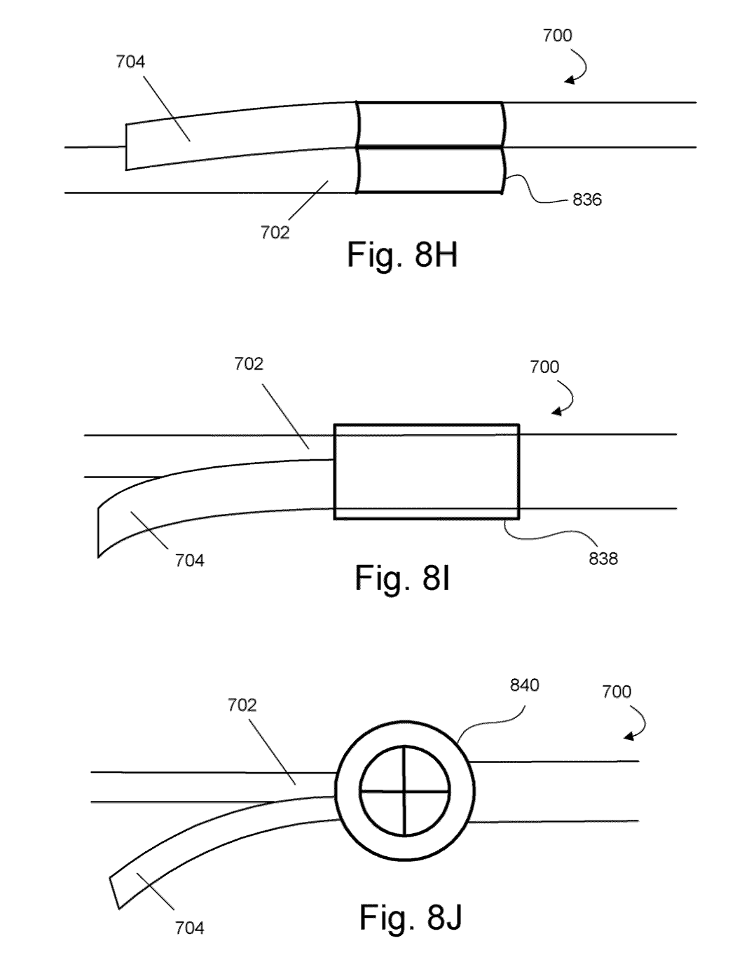

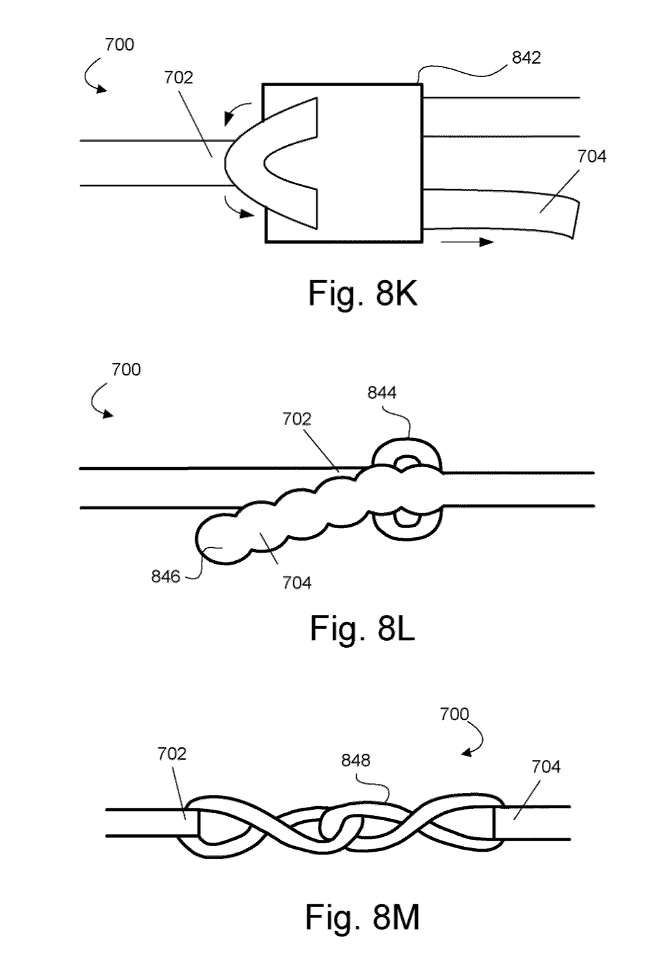

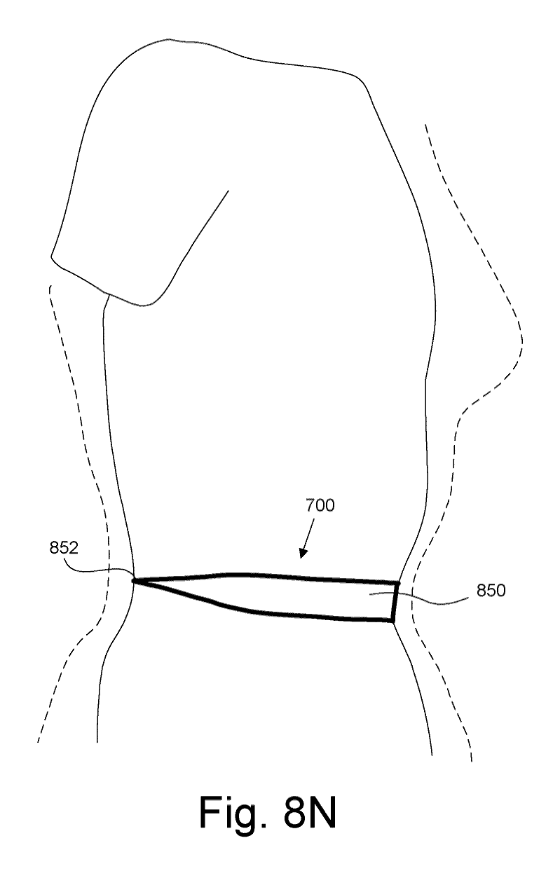

The ends 702, 704 of the band 700 can be configured to be fastened, tied, clipped, clamped and/or otherwise connected to each other to secure the band 700 to the body. Referring now to FIG. 8A, in one embodiment, the band 700 is a rectangular strip of material which does not include any fastening devices on the ends 702, 704. The ends 702, 704 of the band 700 in this embodiment can be connected by tying the ends 702, 704 together or stitching the ends 702, 704 together. In an embodiment, the band 700 can include marks 806 and/or numbers 808 to measure the circumference of the waist.

FIGS. 8B through 8N illustrate various examples of fasteners, hooks, clips, adjusters and/or clamps which can be used to connect the ends 702, 704 of a band 700 together. FIG. 8B illustrates a band 700 having hooks 810 on the first end 702 and eyes 812 on the second end 704 of the band 700. FIG. 8C also illustrates a band 700 having hooks 814 on the first end 702 and eyes 816 on the second end 704 of the band 700. FIG. 8D illustrates a band 700 having a buckle 818 on the first end 702, wherein the second end 704 of the band 700 can be secured to the buckle 818. FIG. 8E illustrates a band 700 having a plurality of apertures 820 on the first end 702 and a loop 822 including fasteners 824 to fit inside the apertures 820 on the second end 704. FIG. 8F illustrates a band 700 having a loop 826 having a tightening device 828 on the first end 702, wherein the second end 704 of the band 700 can be secured to the first end 702 within the loop 826 using the tightening device 828. FIG. 8G illustrates a band 700 having a hinge 830, a band cover 832 and a tightening device 834 on the first end 702, wherein the second end 704 of the band 700 can be secured to the first end 702 under the band cover 832. FIG. 8H illustrates a band 700 having a crimper sleeve 836 to connect the first end 702 to the second end 704 of the band 700. FIG. 8I illustrates a band 700 having a stop sleeve 838 to connect the first end 702 to the second end 704 of the band 700. FIG. 8J illustrates a band 700 including a buckle 840 to connect the first end 702 to the second end 704 of the band 700. FIG. 8K illustrates a band 700 having a stop sleeve 842 to connect the first end 702 to the second end 704 of the band 700. FIG. 8L illustrates a band 700 including a clamp 844 on the first end 702 of the band 700 and built in striations 846 on the second end 704 of the band 700, wherein the second end 704 of the band 700 can be secured to the first end 702 by the clamp 844. FIG. 8M illustrates a band 700 including jack chain connectors 848 on both the first end 702 and the second end 704 of the band 700. FIG. 8N illustrates a band 700 wherein the front portion 850 of the band 700 is a rectangular strap and the rear portion 852 of the band 700 is a thin strap or wire 852. The strap 852 can easily be tightened or released. It is envisioned that the band 700 may include any other fastening devices that would be obvious to one skilled in the art without deviating from the scope of the invention.

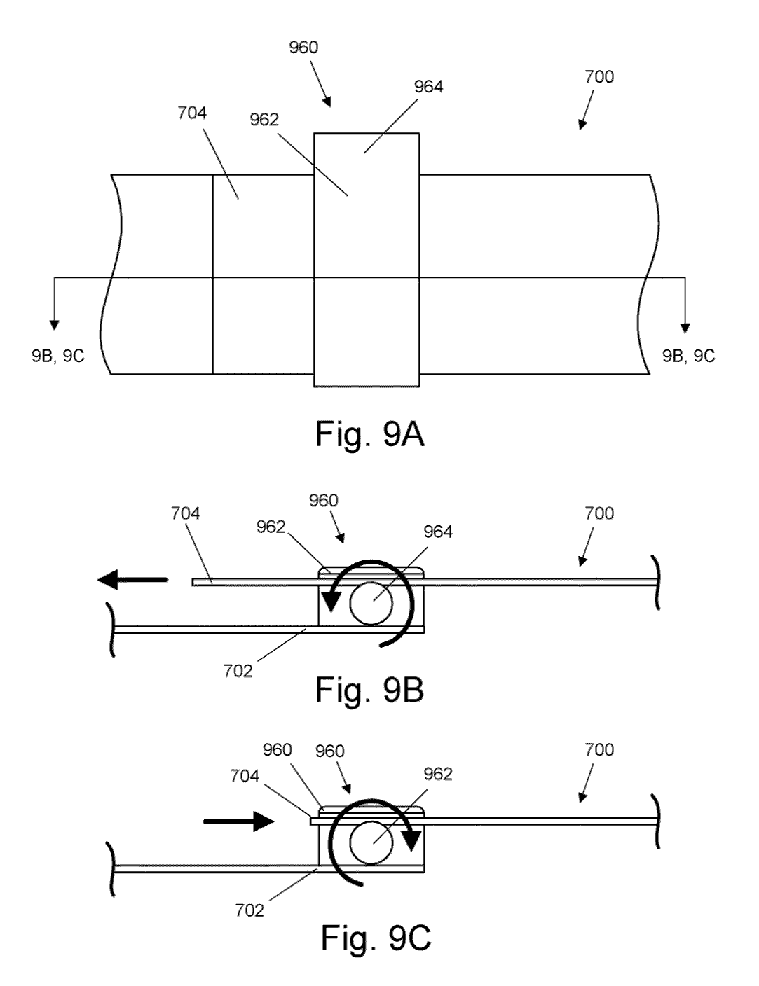

FIGS. 9A through 12B illustrate various examples of adjusters which can be used to connect the ends 702, 704 of a band 700 together while allowing remote adjustment of the circumference of the implanted band 700 (i.e. also referred to herein as the effective length). FIG. 9A illustrates a band 700 having the first end 702 fixedly attached with a housing 962 of an adjuster 960. The second end 704 of the band is threaded into an opening of the housing 962 and held in place within the housing 962 by a shaft 964 that is rotatable by a motor. In some embodiments, the shaft 964 can be an external rotor of the motor. The second end 704 can be held in place, for example, by way of friction, or by way of complementary features such as sprockets (not shown) that rotate with the shaft 964 and mesh with perforations (not shown) along the second end 704. Alternatively, some other features can be used to generate relative movement between the first end 702 and the second end 704, such as complementary teeth resembling a rack and pinion assemblage.

Referring to FIG. 9B, when current is applied to the motor so that the shaft 964 rotates counter-clockwise in the plane of the page, the second end 704 is urged to the left, reducing the circumference of the band 700 to thereby cinch the band 700 around the abdominal wall. The shaft 964 can be limited in its rotation count so that a minimum effective length of the band 700 can be predefined. The effective length can either be preset at two effective lengths (cinched and slackened), multiple effective lengths, or alternatively can be adjusted to any effective length desired by the patient between the minimum effective length and a maximum effective length. The maximum effective length avoids decoupling the second end 704 from the housing 962. Further, the ability of the motor to drive the shaft 964 can be torque limited to avoid accidental over-cinching which can be unsafe and/or uncomfortable to the patient.

Referring to FIG. 9C, the shaft 964 can be released and force applied by the abdominal wall can rotate the shaft 964 clockwise in the plane of the page, allowing the effective length of the band 700 to expand by some predefined amount, for example back to the maximum effective length. The motor of the adjuster 960 can then be reengaged to cinch the band 700 to a new effective length. Alternatively, the motor can be designed to rotate the shaft 964 in either direction.

In other embodiments, the adjuster 960 need not be fixedly attached to the first end 702, but rather can be threaded into an opening of the housing 962 opposite the second end 704, and engaged by the shaft 964 so that the shaft 964 causes both ends 702, 704 to move in opposite directions simultaneously.

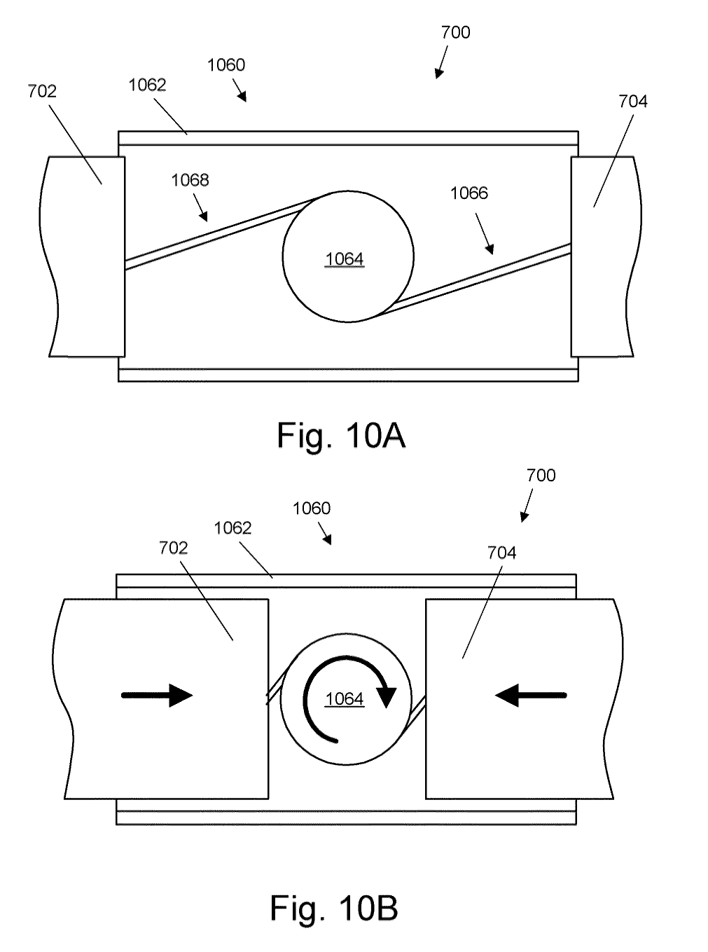

FIG. 10A illustrates an alternative embodiment of an adjuster 1060 wherein the shaft 1064 is arranged so that the axis of rotation is perpendicular to the abdominal wall (i.e., perpendicular to the plane of the page in FIG. 10A). The first end 702 and second end 704 are connected to the shaft 1064 by drawstrings 1066, 1068. The drawstrings 1066, 1068 can be fabricated from the same material as the band 702, or a different biocompatible, nonresorbable and nonbiodegradable material. For example, it may be desirable to fabricate the drawstrings 1066, 1068 from a material that has a higher tensile strength so that a thickness of the drawstrings 1066, 1068 can be narrow. The length of the drawstrings 1066, 1068 should be such that they are fully enclosed in a housing 1062 to distribute cinching force across a surface of the abdominal wall and avoid cutting into the abdominal wall.

Referring to FIG. 10B, when current is applied to the motor so that the shaft 964 rotates clockwise in the plane of the page, the first end 702 is urged to the right and the second end 704 is urged to the left, reducing the circumference of the band 700 to thereby cinch the band 700 around the abdominal wall. As above, the shaft 1064 can be limited in its rotation count so that a minimum effective length of the band 700 can be predefined. The effective length can either be preset at two effective lengths (cinched and slackened), multiple effective lengths, or alternatively can be adjusted to any effective length desired by the patient between the minimum effective length and a maximum effective length, defined by the length of the drawstrings 1066, 1068. Because the ends 702, 704 of the band do not overlap in this embodiment, the ability of the motor to drive the shaft 1064 is limited.

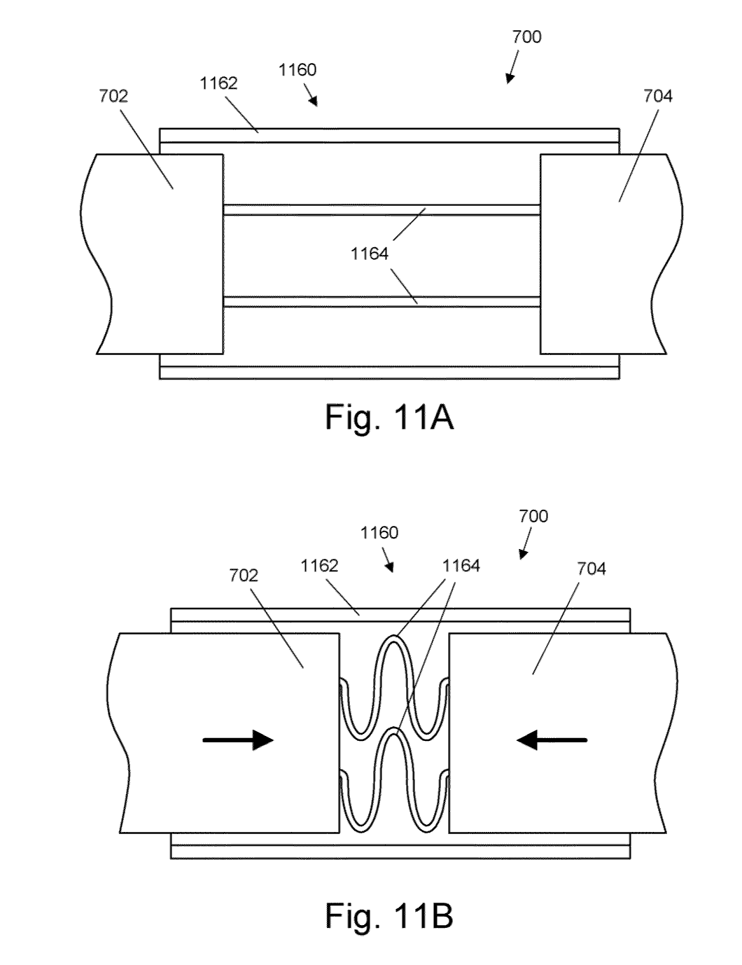

FIGS. 11A and 11B illustrate an alternative embodiment of an adjuster 1160 wherein the first end 702 and second end 704 of the band 700 are connected one or more (two as shown) drawstrings 1164 formed of a shape-memory material. The band 700 has an effective slackened length defined by the cold shape of the shape-memory material (FIG. 11A) and an effective cinched length defined by the hot shape of the shape-memory material (FIG. 11B). The shape-memory material can comprise, for example, a nickel titanium alloy (nitinol). Alternatively, the hot shape and the cold shape can be reversed. The temperature at which the nitinol remembers its high temperature form when heated can be adjusted by slight changes in alloy composition and through heat treatment. The shape recovery process occurs over a range of just a few degrees and the start or finish of the transformation can be controlled to within a degree or two if necessary. It is therefore possible to adjust the effective length of the band 700 by heating the drawstrings 1164 by just a few degrees. The drawstrings 1164 are shown enclosed within a housing 1162 to avoid pinching tissue during the transformation process and to retain heat applied to the drawstrings 1164 and avoid causing any awareness by the patient of the slight temperature increase. The drawstrings can be heated by wires, for example connected to a battery or capacitor, which can be charged (or recharged) remotely, as described in further detail below.

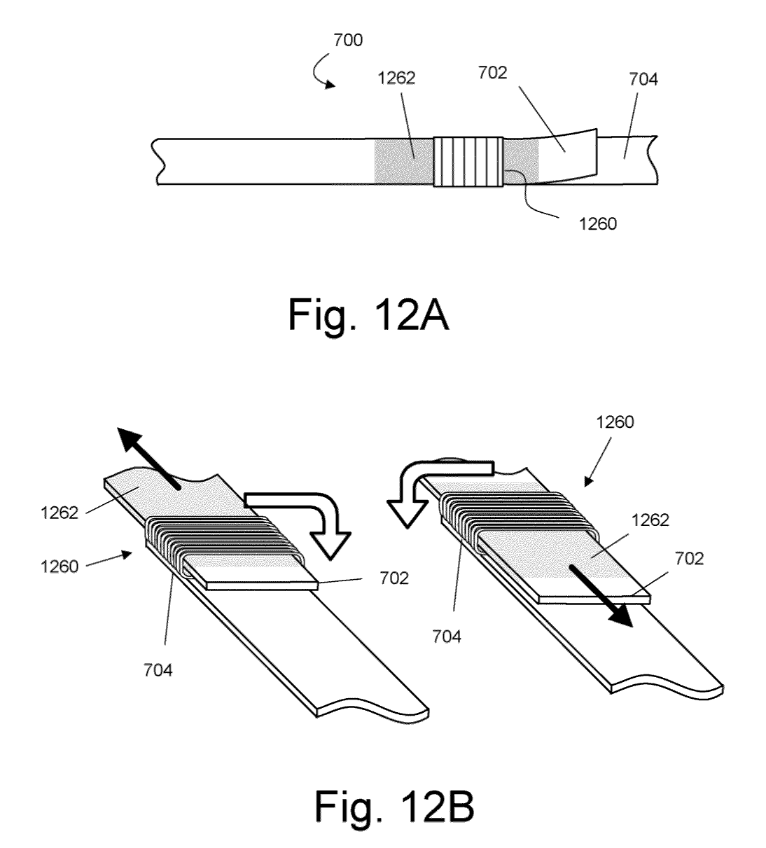

FIG. 12A illustrates a band 700 having the second end 704 fixedly attached with an adjuster 1260 comprising a solenoid having a series of winds. The first end 702 of the band is threaded into the solenoid and held in place, for example by a clamp (not shown) actuated in concert with the solenoid. A portion 1262 of the first end 702 comprises a paramagnetic material. The minimum effective length can be defined by the length of the portion. When current is applied to the solenoid, a magnetic field is generated that acts on the paramagnetic portion of the first end 702. As shown in FIG. 12B, if the current is driven through the solenoid in a clockwise direction, the first end is urged in a direction that increases the effective length of the band 700. If the current is driven through the solenoid in a counter-clockwise direction, the first end is urged in a direction that decreases the effective length of the band 700. By selectively applying current to the solenoid, and clamping the first end in place in the absence of applied current to the solenoid, the effective length can be selectively chosen along the paramagnetic portion 1262 of the first end 702.

FIGS. 9A-12B are intended to reflect the wide variety of mechanisms that can be used to cinch and slacken a band. FIGS. 9A-12B are not intended to be limiting, but rather exemplary. Upon reflecting on the teachings herein, one of ordinary skill in the art will appreciate the myriad different mechanisms that can be used to cinch or slacken a band.

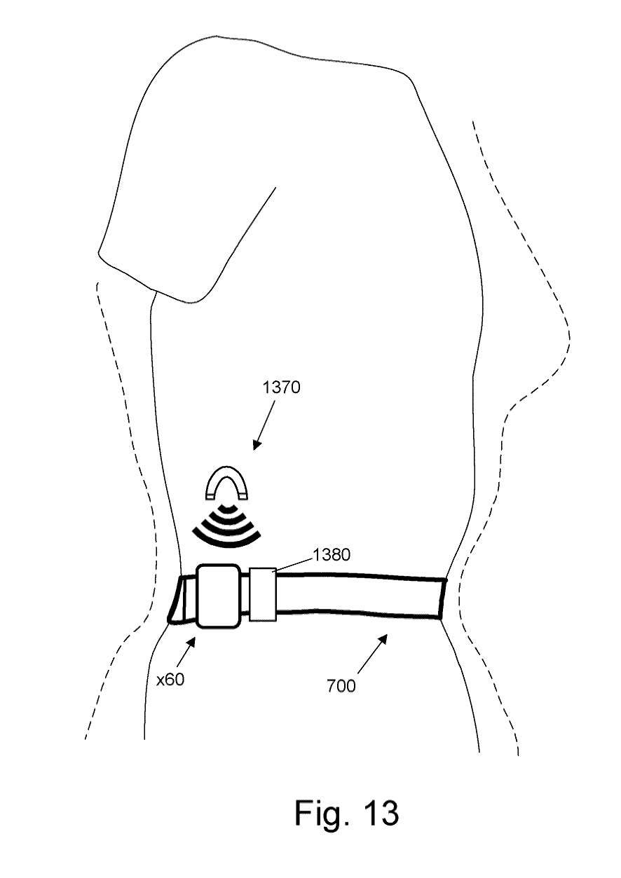

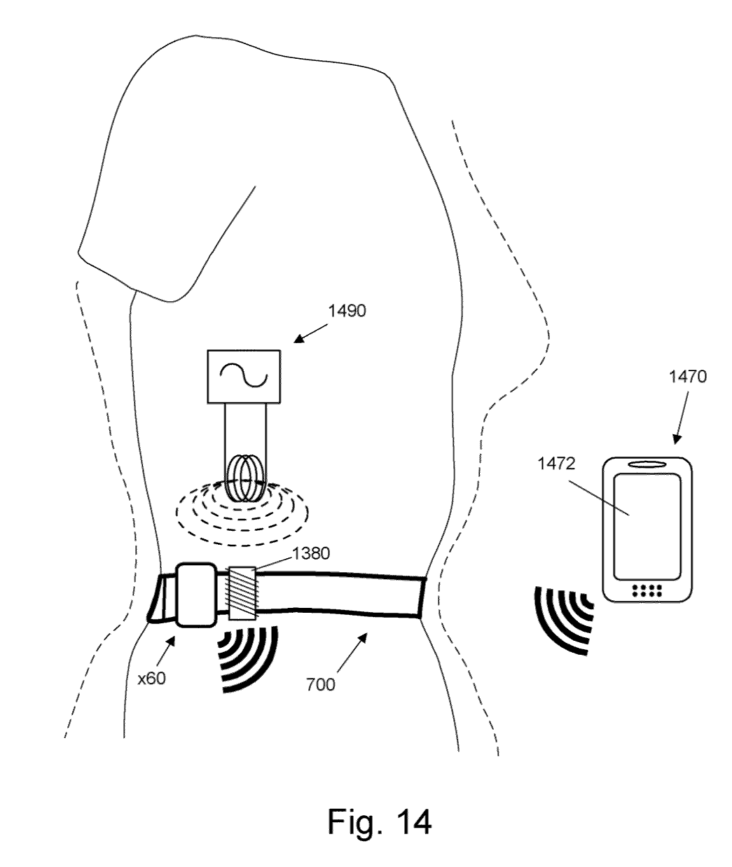

FIG. 13 illustrates an embodiment of a system usable with adjusters and bands to be implanted around an abdomen for waistline reduction allowing for remote adjustment of the effective band length. Adjusters 960, 1060, 1160, 1260 (collectively referred to as x60 in FIGS. 13 and 14) such as shown in FIGS. 9A-12B rely on a power source 1380 to drive the adjustment mechanism. The power source 1380 can be, for example, a battery. Lithium batteries are widely used in medical applications such as artificial pacemakers, and techniques are known for safely implanting such batteries within the body. As shown, actuation of the adjustment mechanism can be achieved, in simplest form, by a magnet placed in the appropriate location to close a reed switch of a simple circuit. The magnet can be placed over the location, driving the adjustment mechanism, until a desired effective length is achieved. Once the effective length is achieved, the magnet can be removed, causing the reed switch to open, thereby disconnecting the adjustment mechanism from the power source and/or control circuit. Cinching adjustments and slackening adjustments can be made by placing the magnet over different locations, closing different reed switches that cause different responses by the adjustment mechanism. Alternatively, the system can include a microcontroller, for example contained in the housing of the adjusters 960, 1060, 1160, 1260. Magnet detection circuitry, coupled to the microcontroller can detect when a magnet is placed over the magnet detection circuitry. Different magnetic fields can be detected by the magnet detection circuitry, causing the adjusters 960, 1060, 1160, 1260 to respond based on the applied magnetic field.

FIG. 14 illustrates an alternative embodiment of a system usable with adjusters and bands to be implanted around an abdomen for waistline reduction allowing for remote adjustment of the effective band length. The system includes a micro-controller associated with the adjusters 960, 1060, 1160, 1260 that is capable of communicating with an external device, for example using telemetry. Operating instructions may be non-invasively provided to the micro-controller through a telemetry circuit in telemetric communication by way of a communication link with an external device 1470, such as a programmer or local transceiver. The telemetry circuit further allows information relating to the state of the system to be sent to the external device 1470 through an established communication link. For example, battery life can be communicated by the micro-controller to the external device 1470 and displayed on a screen 1472. Additionally, the system may include sensors for measuring parameters of the band 700. For example, the system can include a strain gage to measure band tension which can then be communicated to the external device 1470 via the micro-controller. Additionally, the system can communicate warnings for failures. For example, if an adjustment mechanism fails to respond to commands, or where a clamp applied to retain the band 700 at an effective length fails a warning can be sent. FIG. 14 further illustrates a technique for recharging a battery or capacitor for powering the adjustment mechanism. An inductive source coil 1490 can be placed in proximity to a pick-up coil 1380 and the battery or capacitor is charged. Such techniques are commonly used in medical implants, such as fully implantable hearing devices.

As shown in FIGS. 13 and 14, the adjusters 960, 1060, 1160, 1260 are implanted at a location near the posterior. However, the adjusters need not be implanted in the location shown. The adjusters can be implanted in a location deemed desirable by a patient and/or the patient's physician. Factors that may influence positioning include interference of the adjuster with movement/sitting, comfort, visibility, and the planarity or curvature of the location.

While embodiments and applications of this invention have been shown and described, it would be apparent to those skilled in the art having the benefit of this disclosure, that many more modifications than mentioned above are possible without departing from the inventive concepts described herein.

1. A method of adjusting a circumference of an abdominal band implanted subcutaneously for waistline reduction, which comprises:
using an adjustment mechanism associated with the abdominal band and adapted to adjust the circumference of the abdominal band; and
remotely commanding the adjustment mechanism to adjust the circumference of the abdominal band.

2. The method of claim 1, wherein remotely commanding the adjustment mechanism includes applying a magnetic field to actuate the adjustment mechanism.

3. The method of claim 1, wherein remotely commanding the adjustment mechanism includes establishing a telemetric communication link between the abdominal band and an external device and communicating a command by way of the telemetric communication link that actuates the adjustment mechanism.

4. The method of claim 1, wherein adjusting the circumference of the abdominal band includes cinching the abdominal band.

5. The method of claim 4, wherein:
the abdominal band has a first end and a second end and an effective length that defines the circumference of the abdominal band, the effective length spanning at least a portion of the length between the first end and the second end;
the adjustment mechanism is a motor driven shaft that urges the first end and the second end relative to one another; and
cinching the abdominal band is achieved by commanding the motor to rotate the shaft such that the effective length is reduced.

6. The method of claim 4, wherein:
the abdominal band has a first end and a second end;
the adjustment mechanism is one or more draw strings formed from shape memory material and connected between the first end and the second end;
cinching the abdominal band is achieved by heating the one or more drawstrings so that the one or more drawstrings transform from a first expanded shape to a second contracted shape.

7. The method of claim 1, wherein adjusting the circumference of the abdominal band includes slackening the abdominal band.

8. The method of claim 7, wherein slackening the abdominal band includes allowing the abdominal band to be expanded by the abdominal wall.

9. The method of claim 7, wherein:
the abdominal band has a first end and a second end and an effective length that defines the circumference of the abdominal band, the effective length spanning at least a portion of the length between the first end and the second end;
the adjustment mechanism is a motor driven shaft that urges the first end and the second end relative to one another; and
cinching the abdominal band is achieved by commanding the motor to rotate the shaft such that the effective length is increased.

10. An abdominal band for subcutaneously reducing a person's waistline, comprising:
a band adapted to be subcutaneously implanted around a person's abdomen, wherein the band includes an adjustment mechanism for adjusting the circumference of the abdominal band to thereby reduce the person's waistline;
wherein the adjustment mechanism is actuatable remotely by an external device; and
wherein said band is biocompatible, nonresorbable and nonbiodegradable.

11. The abdominal band of claim 10, wherein:
the band has a first end and a second end and an effective length that defines the circumference of the abdominal band, the effective length spanning at least a portion of the length between the first end and the second end; and
the adjustment mechanism is a motor driven shaft that urges the first end and the second end relative to one another.

12. The abdominal band of claim 10, wherein:
the band has a first end and a second end;
the adjustment mechanism is one or more draw strings formed from shape memory material and connected between the first end and the second end.

13. The abdominal band of claim 10, further comprising:
a microprocessor;
a telemetry circuit;
a power source adapted to provide power to the microprocessor, the telemetry circuit, and the adjustment mechanism;
wherein the microprocessor is adapted to communicate with the external device by way of the telemetry circuit; and
wherein the microprocessor is adapted to actuate the adjustment mechanism based on the communication with the external device.

14. The abdominal bad of claim 13, further comprising:
a pick-up coil connected with the power source;
wherein the power source is chargeable by exposing the pick-up coil to an inductive field generated by an external inductive source coil.

15. The abdominal band of claim 10, further comprising:
a microprocessor;
a magnet detection circuit;
a power source adapted to provide power to the microprocessor, the magnet detection circuit, and the adjustment mechanism;
wherein the microprocessor is adapted to communicate with the external device by way of a magnetic field generated by the external device and detected by the magnet detection circuit; and
wherein the microprocessor is adapted to actuate the adjustment mechanism based on the communication with the external device.

16. A system for subcutaneously reducing a person's waistline, comprising:
an abdominal band adapted to be subcutaneously implanted around a person's abdomen including:
a band includes an adjustment mechanism for adjusting the circumference of the band to thereby reduce the person's waistline,
wherein said band is biocompatible, nonresorbable and nonbiodegradable
a power source, and
a pick-up coil connected with the power source;
an external device adapted to communicate with the abdominal band;
wherein the adjustment mechanism is actuatable remotely by an external device; and
an external inductive source coil adapted to generate an inductive field to charge the power source when the pick-up coil is exposed to the inductive field.

17. The system of claim 16, wherein the abdominal band further includes:
a microprocessor;
a telemetry circuit;
wherein the microprocessor is adapted to communicate with the external device by way of the telemetry circuit; and
wherein the microprocessor is adapted to actuate the adjustment mechanism based on the communication with the external device.

18. The system of claim 16, wherein the abdominal band further includes:
a microprocessor;
a magnet detection circuit;
wherein the microprocessor is adapted to communicate with the external device by way of a magnetic field generated by the external device and detected by the magnet detection circuit; and
wherein the microprocessor is adapted to actuate the adjustment mechanism based on the communication with the external device.

19. The system of claim 16, wherein:
the band has a first end and a second end and an effective length that defines the circumference of the abdominal band, the effective length spanning at least a portion of the length between the first end and the second end; and
the adjustment mechanism is a motor driven shaft that urges the first end and the second end relative to one another.

20. The system of claim 16, wherein:
the band has a first end and a second end;
the adjustment mechanism is one or more draw strings formed from shape memory material and connected between the first end and the second end.
